(19) United States
(12) Patent Application Publication
(10) Pub. No.: US 2008/0275479 A1
(43) Pub. Date: Nov. 6, 2008

(54) ANASTOMOTIC SEAL LOADING TOOL

(76) Inventors: Albert K. Chin, Palo Alto, CA (US); Arthur M. Lin, Fremont, CA (US); Alfredo R. Cantu, Pleasanton, CA (US); Hung Pham, San Jose, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/113,547

(22) Filed: May 1, 2008

Related U.S. Application Data
(60) Provisional application No. 60/927,752, filed on May 4, 2007.

(51) Int. Cl. A61B 17/11

(52) U.S. Cl. 606/153; 606/155

(57) ABSTRACT

Described herein are methods and devices for loading an anastomotic seal into a delivery lumen. A loading device can fold the seal into a configuration for insertion into the delivery lumen. For example, the loading device can fold the seal into a prolate spheroidal shape. The delivery lumen can then be inserted into the loading device and mated with the folded seal.

This application claims priority to Provisional Application Ser. No. 60/927,752 entitled “Anastomotic Seal Loading Tool” filed May 4, 2007, which is incorporated herein by reference.

### BACKGROUND OF THE INVENTION

Contemporary coronary artery bypass grafting surgery is performed on a beating heart to reduce complications commonly associated with the prior surgical practice of transitioning a patient onto and off of a heart-lung machine. Performing an aortotomy and a proximal anastomisis on an aorta that is perfused with pressurized blood can be facilitated with temporary sealing methods to curtail blood flow through an aortic aperture. Side-bite and surface-oriented clamping mechanisms have been used to diminish blood loss during such procedures, but such temporary occlusion devices can damage the endothelium and dislodge emboli that may migrate through the circulatory system. Alternative schemes for performing an aortotomy and limiting loss of blood include introducing a plug or seal at the site of the aortotomy, but such schemes commonly inhibit convenient and rapid completion of the graft anastomosis.

In response, new methods and instrumentation have been developed. In accordance with these advancements, an aorto-coronary bypass graft is performed by puncturing the aortic wall and inserting a hemostatic sheath that selectively delivers and positions a seal within the aortic hole. The seal is retented against the aortic wall under tension established by an external structure. The suture anastomosis is performed with the hemostatic seal in place and with a central stem of the seal residing near the location of the last placed stitch. The seal is then removed as a tear-away strip that is pulled, initially by the seal stem, through a removal instrument. Additional discussion of these procedures and instrumentation can be found in U.S. Pat. No. 6,814,743 and application Ser. Nos. 10/123,470 and 10/952,392. These disclosures are also incorporated herein by reference.

The seal, in its fully deployed shape, is too large to easily fit through the aortic hole or in the delivery sheath through which it is delivered. Therefore, use of the seal requires it be manually rolled and partially inserted into the delivery tube immediately prior to insertion into the aortotomy. The seal, which is usually formed of a polyurethane coated surgical suture wound in a spiraling fashion and heat molded to retain its shape, is easily cracked or permanently deformed during manipulation, rendering it unuseable. Further, the seal is generally not packaged and provided pre-loaded in the delivery tube, as such pre-packaging can result in deformation of the seal and result in improper deployment.

Accordingly, current surgical procedures could benefit from improved techniques and devices for folding the hemostatic seal.

### SUMMARY OF THE INVENTION

In accordance with the invention, a hemostatic seal loading apparatus and method of use is disclosed. The loading apparatus has a receiving area for receiving an unfolded, or substantially unfolded, hemostatic seal and two seal contact surfaces, on either side of the receiving area, capable of converging in relation to one another and folding the seal therebetween. The apparatus herein described prepares the seal for insertion into a delivery sheath and subsequent deployment into an aortic hole, while reducing the risk of seal damage that can result from manual manipulation.

A further object of this invention is to provide a surgeon with a properly folded hemostatic seal in the operating room, while avoiding the seal deformation associated with pre-folding the seal.

A further object of this invention is to provide instrumentation and a reliable method by which to insert a folded seal into a delivery tube.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a perspective view of one exemplary embodiment of a system described herein.

FIG. 2 is a top and side view of one embodiment of a sealing element.

FIG. 3 is a front view of one embodiment of a device described herein.

FIGS. 4(a)-(c) are front views of one embodiment of a system described herein.

FIG. 4(d) is a perspective view of one embodiment of a sealing element.

FIG. 5(a) is a side view of another embodiment of a device described herein.

FIG. 5(b) is a front view of the device depicted in FIG. 5(a).

FIGS. 5(c)-(e) are side views of another exemplary embodiment of a system described herein.

FIG. 6(a) is a perspective view of another exemplary embodiment of a device described herein.

FIG. 6(b) is a front view of the device depicted in FIG. 6(a).

FIGS. 6(c)-(e) is a top view of an exemplary embodiment of a system described herein.

FIG. 7(a) is a perspective view of another embodiment of a system described herein.

FIGS. 7(b)-(c) are front views of the system depicted in FIG. 7(a).

FIGS. 8(a)-(c) are front views of another embodiment of a system described herein.

FIGS. 9(a)-(d) are perspective views of another embodiment of a system described herein.

FIG. 10(a) is a perspective view of another embodiment of a system described herein.

FIGS. 10(b)-(d) are front views of the system depicted in FIG. 10(a).

FIGS. 11(a)-(c) are front views of an exemplary embodiment of a system described herein.

FIGS. 12(a)-(c) are front views of another embodiment of a system described herein.

FIGS. 13(a)-(c) are front views of another embodiment of a system described herein.

### DESCRIPTION OF EXEMPLARY EMBODIMENTS

Disclosed herein are various systems and devices for loading a hemostatic seal into an insertion instrument. Generally, the systems can include a hemostatic sealing element, a seal loading device, and a seal insertion instrument. In one aspect, the seal loading device can fold the sealing element into a reduced size for insertion into a delivery area of the seal insertion instrument. In another aspect, the seal loading device and seal insertion instrument can be used cooperatively to insert the sealing element into the seal insertion instrument.

Hemostatic sealing elements are generally known in the art and find use during cardiac procedures, such as coronary by-pass procedures, for sealing an aperture created in either an off-pump (beating) or on-pump (non-beating) heart. Such devices are generally folded in the operating room, deployed through a tissue aperture, and expanded within the heart to create a seal. Rather than pre-package the sealing elements in a folded configuration, a surgeon folds the hemostatic seal in the operating room, shortly before inserting the sealing element into a seal insertion instrument. In particular, a surgeon will hand roll the sealing element and manually insert it into the insertion instrument. Unfortunately, this can be a delicate procedure, sometimes resulting in several broken seals before proper folding and loading is achieved. The system disclosed herein utilizes a seal loading device that mechanizes the task of folding the sealing element and/or inserting it into the insertion instrument, thereby reducing the amount of time necessary to prepare the seal for use and the risk of sealing element damage resulting from improper manual manipulation.

Reference will now be made in detail to the exemplary embodiments of the invention, examples of which are illustrated in the accompanying drawings. Wherever possible, the same reference numbers will be used throughout the drawings to refer to the same or like parts.

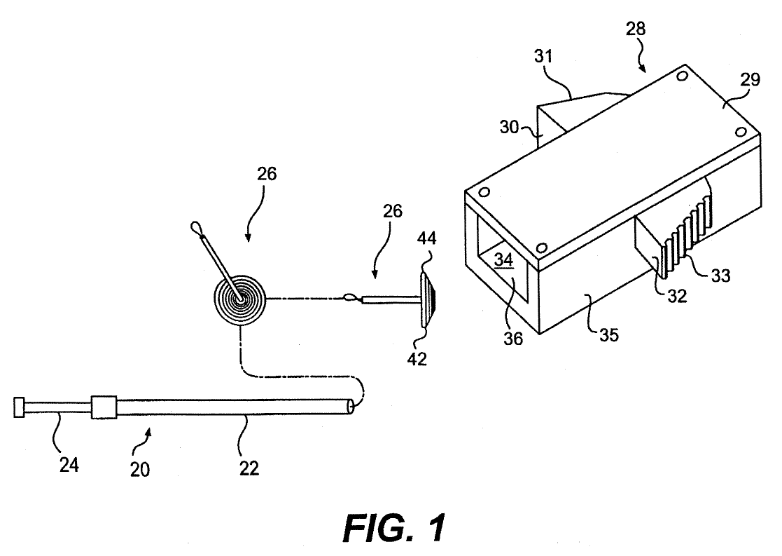

Referring now to FIG. 1, there is shown a seal insertion instrument 20, a hemostatic sealing element 26, and an embodiment of the seal loading apparatus 28. Seal insertion instrument 20 includes a sheath 22 of outer diameter sized for insertion into an aortic hole. The insertion instrument can also incorporate a deployment mechanism for selectively ejecting hemostatic sealing element 26 from its confinement within sheath 22 and into the aorta. An example of such a mechanism is a plunger 24, disposed to slide axially within sheath 22.

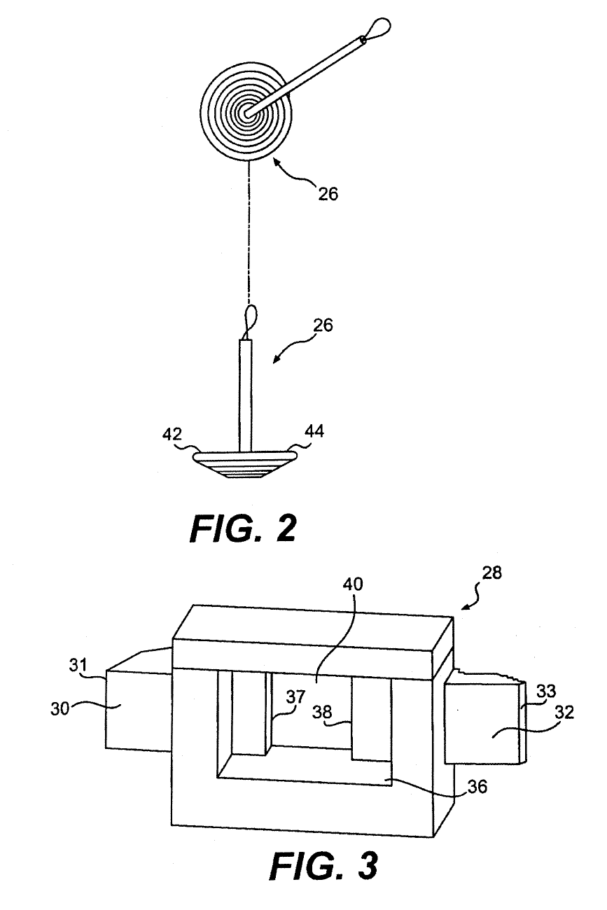

As illustrated in FIGS. 1 and 2, sealing element 26, in its deployed state, is of a convex-concave or mushroom shape with an outer diameter larger than the aortic aperture to be sealed. Medical seals of various shapes, however, can be used with the seal loading apparatus described herein.

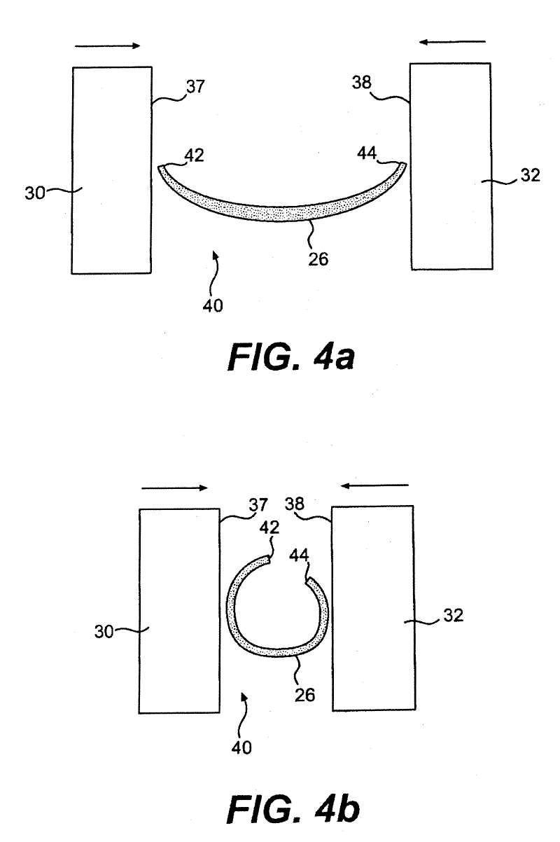

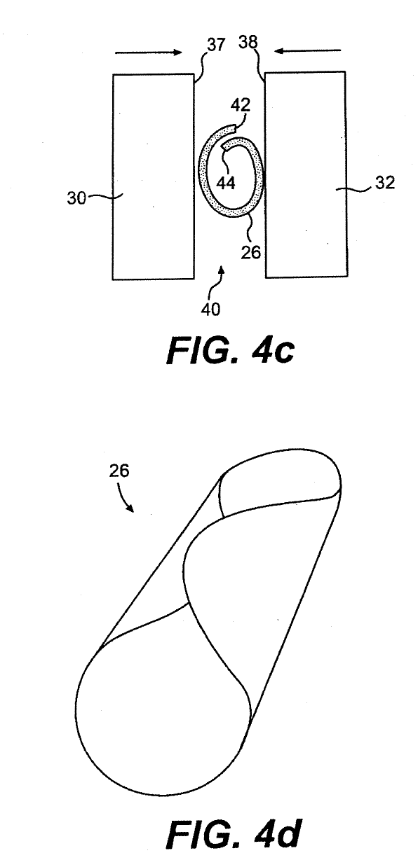

In order to insert sealing element 26 into an aortic aperture, the sealing element can be folded by overlapping any two non-adjacent points along the seal's edge. For example, in one aspect, as illustrated in FIG. 2, edges 42 and 44 are opposing points, spaced by an angle α, of 180 degrees. Alternatively, the points can be spaced at least 90 degrees, and in another aspect, space in the range of about 150 and 180 degrees from one another. This method of folding renders the previously mushroom-shaped sealing element in the shape of a prolate spheroid or ellipsoid with somewhat blunted or flattened ends. In lateral cross-section, the folded seal can be substantially circular. Axially, the cross-section appears generally elliptical with blunted or flattened ends. This configuration is depicted in FIG. 4(d). The terms “prolate spheroid” and “prolate ellipsoid” are not used here in their precise geometric sense. Rather, a section of these shapes represents a portion of the folded sealing element having a generally taco- or football-like shape.

Once folded, seal 26 can be inserted, lengthwise, into an insertion instrument and, subsequently, through an aortic aperture, then released. The fully deployed seal, having thus resumed its mushroom shape, can then be anchored against the aperture through which it was inserted, sealing the aortic hole and preventing blood loss.

In one aspect, sealing element 26 can be formed using a hollow tube of medical grade material such as polyvinyl chloride, PEBAX, or other polymer material. The material can then be extruded about a looped suture or wire or other tensile member for improved tensile strength. Alternatively, a solid, flexible rod of similar material having sufficient tensile strength can be used. The hollow tube (or solid rod) can be helically or spirally wound into the configuration of the mushroom-shaped sealing member, with a central stem formed thereon. The adjacent filaments (tube and/or solid rod) can be lightly adhered together through the application of heat and pressure to a thermoplastic material, or through other suitable adhesive attachments to form the substantially fluid-impervious sealing element that is flexible and resilient for confined packing within the hollow sheath of the seal-insertion instrument. The light adhesion between adjacent filaments can permit disassembly of the sealing element by tearing along the boundary between adjacent filaments under tension applied to the central stem.

Further details regarding sealing element 26 and additional embodiments of the aortic sealing element can be found in U.S. Pat. No. 6,814,743, and U.S. application Ser. Nos. 10/123,470 and 10/952,392, incorporated herein by reference. As mentioned above, however, the invention herein disclosed is not limited to use with any one of these seals and could be used in conjunction with seals of various shapes and sizes.

FIG. 1 also depicts seal loading apparatus 28. Sealing element 26 can be placed inside loading apparatus 28 and the loading apparatus can fold the sealing element into a configuration, for example, as previously described, such that it can be inserted into insertion instrument 20. In one aspect, seal loading apparatus 28 can comprise a housing 29, substantially rectangular in shape and including top and bottom walls, side walls, and ends. The housing structure can be defined as one piece or a plurality of walls fixed in relation to one another. In another embodiment, the housing may not be rectangular or enclosed on all sides. The form factor of the structure can vary in light of numerous considerations, including the size and shape of the sealing element to be folded, ergonomic concerns, or aesthetic considerations.

In one aspect, housing 29 can also have both an inner and outer surface, 34 and 35, respectively. The inner surface defining a space so as to contain a sealing element. In another aspect, there is an opening 36 in one end of the housing. The opening can be sized to receive an unfolded, or substantially unfolded, sealing element, so as to permit insertion of the sealing element into the housing. In the embodiment depicted in FIG. 1, the opposing end of housing 29 is closed, but in other embodiments it can also comprise an opening. Additionally, the opening through which housing 29 receives a sealing element can be located elsewhere in the housing structure and need not be at one of the ends. Further, the housing can have multiple openings through which a sealing element can be inserted or retrieved.

In other embodiments, rather than having openings in either one or more of the top, bottom, side or end walls, one or more of these walls can be entirely absent. Additionally, aside from providing for insertion or retrieval of the sealing element, openings or absent walls in the housing structure could provide means by which to visually inspect the placement of the sealing element within the housing and confirm proper folding of the sealing element. Alternatively, visual confirmation can be achieved through the use of loading apparatus components comprised of a transparent material.

Referring again to FIG. 1, the loading apparatus can further comprise a first and second folding member, 30 and 32, respectively, extending through opposing walls of the housing. In one aspect, folding members 30 and 32 can be selectively and manually convergent in relation to one another. As used herein, the term “convergent” does not require that both folding members be movable in relation to the housing, rather, only that the members move in relation to one another. For example, the first member can be stationary or fixed to the housing, while the second is selectively movable towards the first. In another embodiment of the loading apparatus, rather than two folding members extending through opposing walls of housing 29, one of the folding members can be integrated into the housing. Thus, loading apparatus 28 would comprise only one folding member movable in relation to the housing, but the folding members would remain convergent in relation to one another.

In another aspect of the embodiment depicted in FIG. 1, folding members 30 and 32 may comprise user contact surfaces 31 and 33, respectively, for user interface and/or control of folding members 30 and 32 and seal contact surfaces 37 and 38 for folding the sealing element. For example, the user contact surfaces can be disposed outside the housing, and seal contact surfaces 37 and 38, disposed within the housing. The user contact surfaces 31 and 33 can incorporate a lever, button, or some other actuator to facilitate selective manual manipulation of folding members 30 and 32. The user contact surfaces can also incorporate user-friendly ridges, knurls, or other gripping means.

An alternative embodiment, as previously mentioned, can have one folding member integrated into, or fixedly mated with, the housing. As a result, loading apparatus 28 can only have one user contact surface for control of the loading apparatus.

The seal contact surfaces, 37 and 38, of folding members 30 and 32 are described in more detail below.

FIG. 3 shows a front view of the embodiment of the seal loading apparatus depicted in FIG. 1. Folding members 30 and 32, with seal contact surfaces 37 and 38, respectively, define a channel therebetween serving as a receiving area 40 for receiving an unfolded, or substantially unfolded, sealing element between the folding members.

In one aspect, in order to fold a sealing element in preparation for insertion into a seal insertion instrument, a sealing element is inserted through opening 36 and placed into receiving area 40, between seal contact surfaces 37 and 38. The seal contact surfaces can then selectively converge in relation to one another through manual manipulation of user contact surfaces 31 and 33 of folding members 30 and 32. As the seal contact surfaces converge, each comes into contact with a diametrically opposed edge of the sealing element and exerts a force thereon. In another embodiment, as the seal contact surfaces converge, they come into contact with non-adjacent, but not necessarily diametrically opposed, points along the sealing element edge.

FIGS. 4(a)-(c) depict the manipulation of sealing element 26 as seal contact surfaces 37 and 38 progressively converge. Seal contact surfaces 37 and 38 first contact diametrically opposed edges 42 and 44 of sealing element 26. As the seal contact surfaces continue to converge, seal edges 42 and 44 slide up the respective seal contact surfaces. After still further convergence, edges 42 and 44 begin to curl back towards the center of the receiving area where the edges overlap and seal 26 takes on the prolate spheroid shape described above.

FIG. 4(c) shows a latitudinal cross-section of sealing element 26 in a fold-completed configuration. FIG. 4(d) shows a perspective view of sealing element 26 in a fold-completed configuration such that the prolate spheroid shape may be observed.

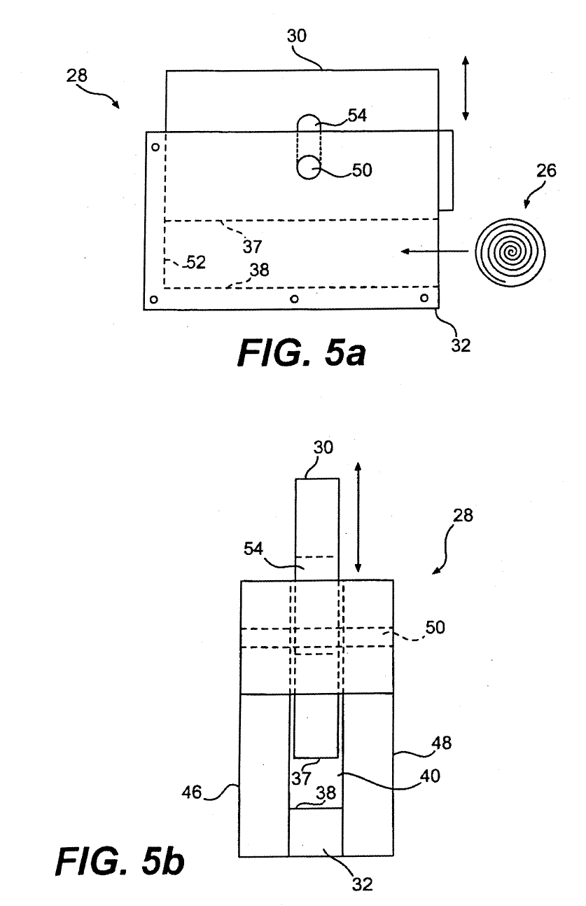

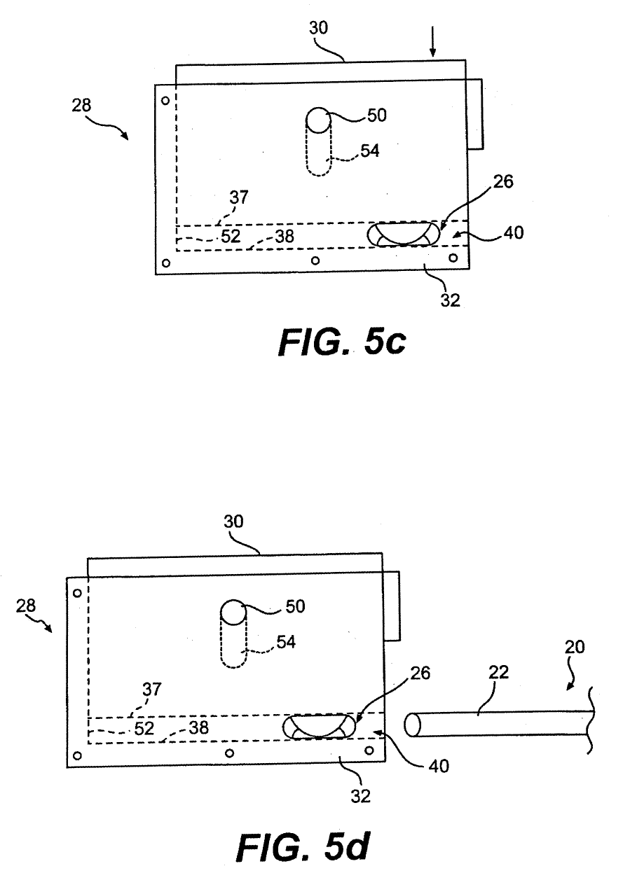

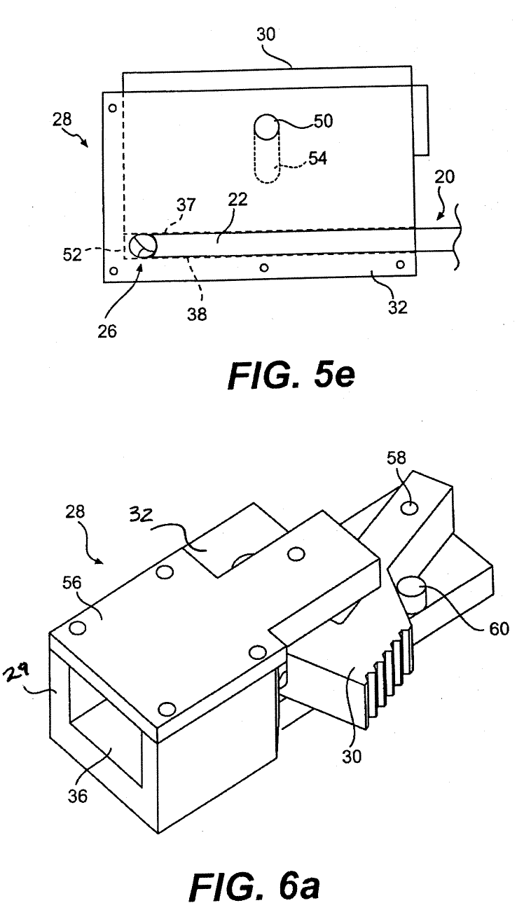

Referring now to FIGS. 5(a) and (b), another embodiment of the seal loading apparatus is shown. In this embodiment, loading apparatus 28 is comprised of folding members 30 and 32, side plates 46 and 48, and pin 50. Folding member 32, having seal contact surface 38 is stationary and fixed between the two side plates. Folding member 30, having seal contact surface 37, is slidably maintained between the two side plates by pin 50 extending through the side plates and slot 54 in folding member 30. It should be appreciated, however, folding member 30 can maintain its sliding relationship with the side plates through a number of methods, such as through guiding grooves, and does not necessarily require pin 50 and slot 54. The sliding connection allows for the selective parallel convergence of folding members 30 and 32.

When, as shown in FIG. 5(b), slidable folding member 30 is positioned away from folding member 32, there exists a channel between seal contact surface 37 and seal contact surface 38 serving as a receiving area 40 for receiving unfolded, or substantially unfolded, sealing element 26. In order to fold a sealing element in preparation for insertion into a seal insertion instrument, an unfolded, or substantially unfolded, sealing element is placed into the receiving area, between seal contact surfaces 37 and 38, as shown in FIG. 5(a). The seal contact surfaces can then selectively converge in relation to one another through manipulation of slidable folding member 30, each seal contact surface initially coming into contact with diametrically opposed edges of the sealing element and exerting a force thereon. Continued convergence folds sealing element 26 such that the opposing edges curl back toward the center of the receiving area and overlap one another, leaving seal 26 in a prolate spheroid shape as shown in FIG. 5(c).

In another aspect, slot 54 is of such length that pin 50 prevents slidable folding member 30 from converging upon folding member 32 within a distance that would permanently deform the sealing element or otherwise prevent the seal from resuming its unfolded shape in the absence of any force acting thereon. In other embodiments, there may exist other known means of so restraining slidable folding member 30. For example, a pin or protrusion extending from folding member 32 or side plate 46 or 48 can inhibit the travel of folding member 30.

Referring to FIGS. 5(d) and (e), folded sealing element 26 can then be at least partially loaded into sheath 22 of seal insertion instrument 20 by inserting the distal tip of the sheath into receiving area 40 and forcing sealing element 26 against an abutment surface 52. The abutment surface can prevent further movement of the sealing element and/or act as a counter force when sheath 22 is pushed against sealing element 26. Once the sealing element is positioned between abutment surface 52 and sheath 22, applying further force to sheath 22 can force the end of the prolate spheroid seal into the distal tip of the sheath.

In one aspect, converging the folding members does not fully fold the sealing element and/or the sealing element is folded (i.e., the sides are overlapped), but the diameter of the sealing element is larger than desired. Further insertion of the sheath will reduce the diameter of the sealing element and more of the prolate spheroid sealing element can be captured within seal insertion instrument 20. Sheath 22 can then be withdrawn from receiving area 40, while retaining the folded sealing element.

Finally, as illustrated in FIG. 5(e), at least a portion of the sealing element is positioned within and detachably mated with sheath 22. The folded sealing element and the sheath can mate, for example, with a friction or interference fit. In addition, only a portion of the sealing element need extend into the inner lumen of sheath 22.

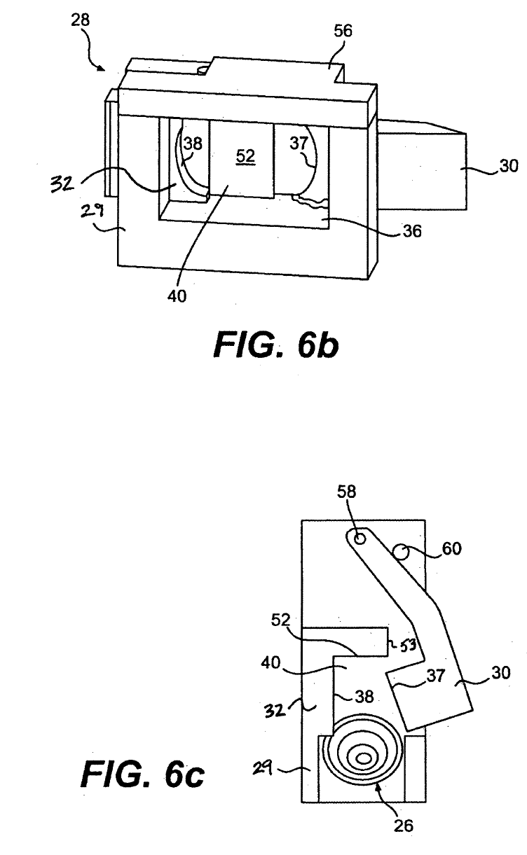

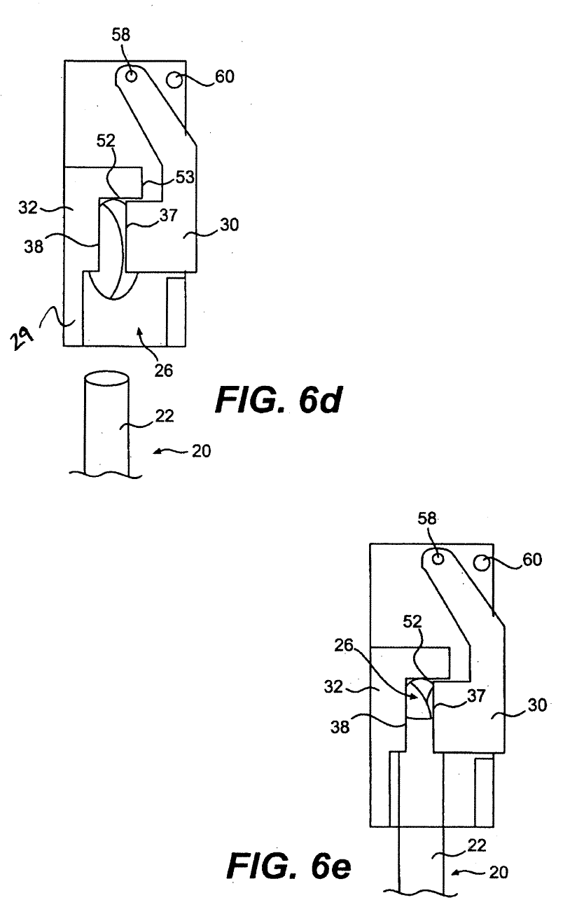

Referring now to FIGS. 6(a) and (b), another embodiment of the seal loading apparatus is shown. In this embodiment, seal loading apparatus 28 can be comprised of housing 29, including, for example, a lower body member and an upper plate 56. Housing 29 together with folding members 30, 32 can define a receiving area for insertion of an unfolded, or substantially unfolded, sealing element. In one aspect, the seal loading apparatus of FIGS. 6(a) and (b) includes one folding member configured to articulate relative to housing 29 and one folding member fixed relative to housing 29. For example, as shown in FIG. 6(b), folding member 32 and seal contact surface 38 are integrated into housing 29, while folding member 30 can articulate relative to housing 29 and folding member 32.

In one aspect, folding member 30 and the housing are pivotally mated within one another. For example, a pivot pin 58, or other pivoting, or jaw-like connection can permit articulation of folding member 30. In another aspect, folding member 30 can be formed integrally with housing 29 and articulate via a living hinge. As used herein, the term “jaws” or “jaw-like connection” is used to describe two bodies or members with substantially opposing surfaces, such as seal contact surfaces 37 and 38 in FIG. 6(b). Similar to the use of the word “convergent,” discussed above, the term “pivotal” or “pivoting,” does not require that both bodies be movable. Rather, only that the members move in relation to one another.

As shown in FIGS. 6(c) and (d), respectively, folding member 30 can move between a first position where the receiving area is configured to receive an unfolded sealing element and a second position where folding member 30 folds the sealing element. In one aspect, folding member 30 is biased in the first position. For example, a spring or living hinge can provide a first position bias. In the absence of forces thereon, the folding member remains in the first position to allow an unfolded, or substantially unfolded, sealing element to be placed into receiving area 40 between the seal contact surfaces 37, 38.

In another aspect, seal loading apparatus 28 can limit the travel of folding member 30. For example, the seal loading apparatus can include stops such that folding member 30 is constrained between the first and/or second positions. A variety of stops can be used, including, for example, pin 60. As folding member 30 moves away from the receiving area, pin 60 contacts the folding member and limits its movement.

In addition, or alternatively, housing 29 can prevent seal contact surface 37 of folding member 30 from converging beyond the second position. For example, housing 29 can comprise motion-limiting surface 53 positioned such that, as folding member 30 pivotally converges upon seal contact surface 38, a portion of the folding member abuts motion-limiting surface 53, thus preventing the two seal contact surfaces from converging more than a predetermined distance. In other embodiments, a motion-limiting surface or stop can be defined by, for example, another portion of housing 29, upper plate 56, or folding member 30.

The spacing of the seal folding members 30, 32 in the second position can be chosen such that when seal folding member 30 moves from the first position to the second position, sealing element 26 is folded. Thus, the spacing of the folding members 30, 32 in the second position can depend, for example, on the desired diameter of the sealing element, the properties of the sealing element, and/or the shape of seal contact surfaces 37, 38. In one aspect, the spacing of the seal contact surfaces in the second position have a distance corresponding to the width of a sealing element folded into a prolate spheroidal shape. In another aspect, the spacing of the seal contact surfaces in the second position provide a channel having a shape at least partially corresponding to a sealing element folded into a prolate spheroidal shape. In yet another aspect, motion-limiting surface 53 is positioned to prevent seal contact surfaces 37, 38 from converging within a distance that would permanently deform or damage the sealing element.

In one embodiment, loading apparatus 28 can provide tactile feedback to a user indicating that the seal folding members have reached the second position. In one aspect, contact of the seal folding member 30 against motion-limiting surface 53 can provide tactile feedback. In another aspect, a snap-fit between the seal folding member and housing 29 can provide tactile feedback. For example, as the seal folding member travels between the first and second position, a protrusion on the inside surface of cover 56 and/or on another portion of the inner surface of housing 29 can mechanically engage the folding member.

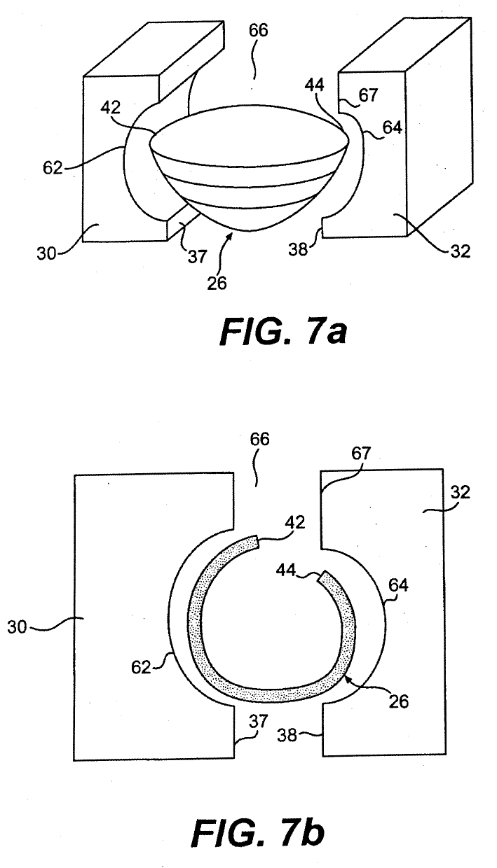

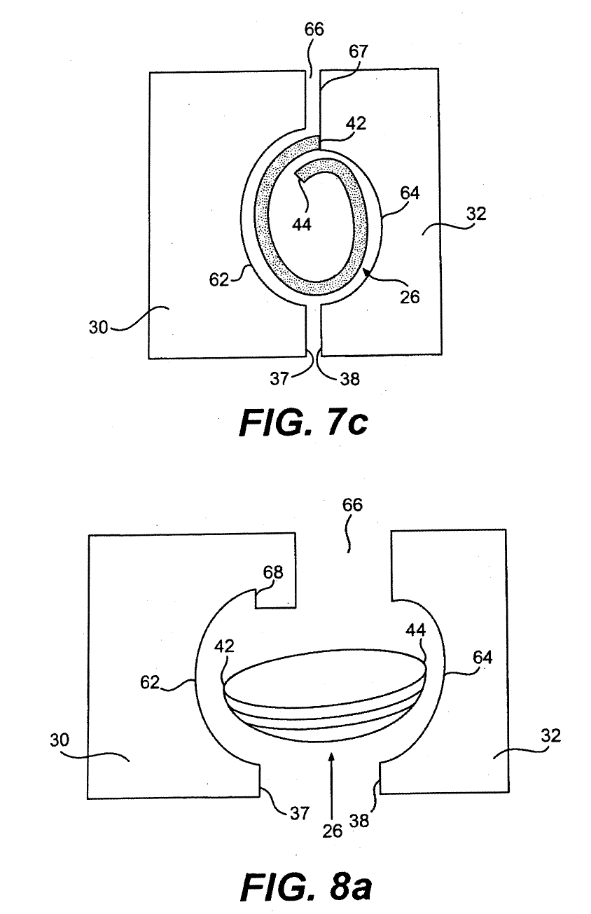

FIG. 6(b) shows that seal contact surfaces 37 and 38 can be curved so as to facilitate the folding of a sealing element as the two surfaces converge upon one another. FIGS. 7(a)-(c), discussed in more detail below, illustrate how curved seal contact surfaces can aid in the folding of a sealing element. The curved seal contact surfaces, and in one aspect, other surfaces of the loading instrument, such as the inner rear wall, inner lower surface, and/or inner upper surface of receiving area 40 can cooperate to facilitate folding of the sealing element. In one such aspect, at the rear of the receiving area, and integrated into housing member 29, can define an abutment surface 52.

Referring now to FIGS. 6(c) and (d), in order to fold a sealing element in preparation for insertion into a seal insertion instrument, an unfolded, or substantially unfolded, sealing element 26 can be placed into receiving area 40, between seal contact surfaces 37 and 38. The seal contact surfaces can then, as a result of an inward force applied to folding member 30, selectively converge in relation to one another, each seal contact surface initially coming into contact with diametrically opposed edges of the sealing element and exerting a force thereon. Continued convergence folds sealing element 26 such that the opposing edges curl back toward the center of the receiving area and overlap one another, leaving seal 26 in a prolate spheroid shape as shown in FIG. 4(d).

Referring now to FIGS. 6(d) and (e), folded sealing element 26 can then be at least partially loaded into sheath 22 of seal insertion instrument 20 by inserting the distal tip of the sheath into receiving area 40 and forcing sealing element 26 against abutment surface 52. Once positioned between abutment surface 52 and sheath 22, further insertion of sheath 22 into receiving area 40 can insert a part or the whole of the prolate-spheroidal-shaped seal into the distal tip of the sheath. Sheath 22 can then be withdrawn from receiving area 40, while retaining the folded sealing element.

In another aspect, in addition to preventing damage to the sealing element as discussed above, motion-limiting surface 53 could serve to prevent folding member 30 from converging upon seal contact surface 38 to an extent that would prevent insertion of sheath 22 of insertion instrument 20 into loading apparatus 28. For example, motion-limiting surface 53 could be positioned so as to ensure the two contact surfaces, 37 and 38, do not converge within a distance less than the outer diameter of sheath 22.

Referring now to FIGS. 7(a)-13(c), several exemplary embodiments of the seal contact surfaces are shown. These configurations are universally applicable to various seal loading apparatuses and can be incorporated into any one of the seal loading apparatus embodiments previously discussed. Further, as mentioned above, where seal contact surface “convergence” is discussed, it should be appreciated the term can refer to situations in which both seal contact surfaces are moving towards one another and situations in which one seal contact surface is stationary, while the opposing surface is moving toward the other.

FIGS. 7(a)-(c) illustrate seal contact surfaces 37 and 38, having opposing curved portions 62 and 64, respectively, and a dynamic channel 66 therebetween. The curved portions of the seal contact surfaces serve to guide diametrically opposed edges of sealing element 26 as the seal contact surfaces converge. For example, as the width of channel 66 is reduced, the sealing element can slide relative to curved portions 62 and/or 64. As the sealing element moves relative to the seal contact surfaces 37 and/or 38, the shape of the seal contact surfaces directs the sealing element into a folded configuration.

In one embodiment, seal contact surfaces 37, 38 are configured to allow both edges 42, 44 to slide relative to the seal contact surfaces. For example, the seal contact surfaces can comprise a low friction surface that permits sliding when pressure is applied the sealing element. Conversely, the seal contact surfaces can be configured to limit movement of the sealing element relative to at least one of the seal contact surfaces 37, 38. In one aspect, one of seal contact surfaces has a higher coefficient of friction (with respect to the sealing element) compared to the other seal contact surface. For example, the seal contact surfaces can be formed of different materials and/or include friction increasing/decreasing coatings. Alternatively, or additionally, as described below, the seal contact surfaces can include a stop that acts as a barrier to inhibit sealing element movement relative to one or both of the seal contact surfaces 37, 38.

In one aspect, the curved portions 62, 64 of seal contact surfaces 37, 38 have generally the same shape and size and are located at corresponding positions on seal contact surfaces 37, 38. Alternatively, the curved portions 62, 64 can have different shapes, sizes, and/or relative locations.

FIGS. 7(a)-(c) illustrate curved portions 62, 64 on opposing seal contact surfaces 37, 38 having different radii. The difference in curvature results in an offset between the top of curved portions 62, 64. As seal contact surfaces 37, 38 converge, the offset directs edge 44 of the sealing element 26 under edge 42 and facilitates overlap and folding of sealing element 26.

In addition, as mentioned above, seal contact surfaces 37 and/or 38 can be configured to limit movement of the sealing element within channel 66. In one aspect, the offset of the curved portions can provide a seal stop surface 67 defined by a portion of seal contact surface 38, adjacent to curved portion 64. For example, seal stop surface 67 sits opposite to a portion of curved portion 62 of seal contact surface 37. As the seal contact surfaces converge in relation to one another and a first seal edge 42 begins to curl back toward the center of dynamic channel 66, first edge 42 abuts seal stop surface 67. The seal stop surface inhibits further movement of first edge 42 in relation to seal contact surface 38 and guides opposed edge 44 under first edge 42.

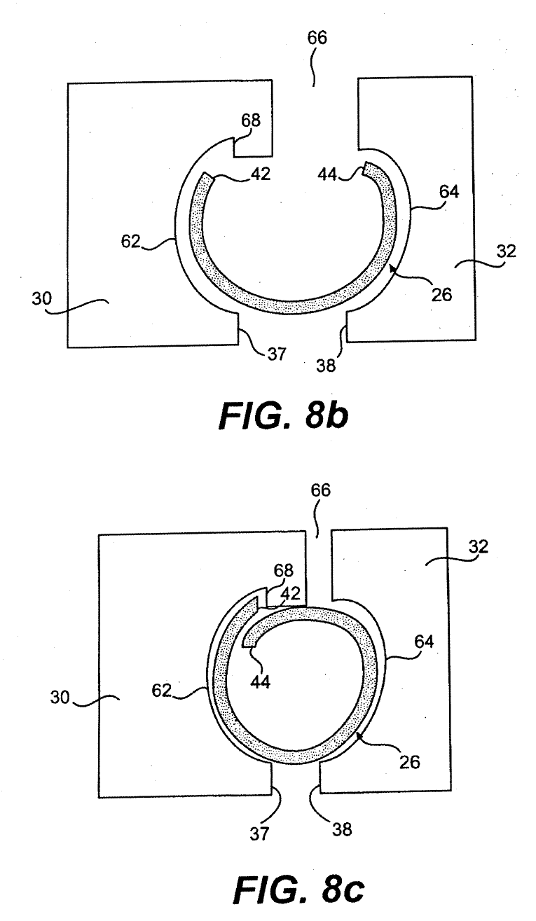

Referring to FIGS. 8(a)-(c), it is also possible to have a seal stop surface within the curved portion of one of the seal contact surfaces. FIG. 8(a) depicts such a configuration. Seal contact surface 37 has a curved portion 62 with a shoulder or lip 68 formed therein. During seal folding, as the seal contact surfaces converge in relation to one another and opposing seal edges curl toward one another, the first seal edge 42 contacts shoulder 68 formed in curved portion 62 and is prevented from further movement in relation to that surface. As the seal contact surfaces continue to converge, opposing seal edge 44, is guided under first seal edge 42.

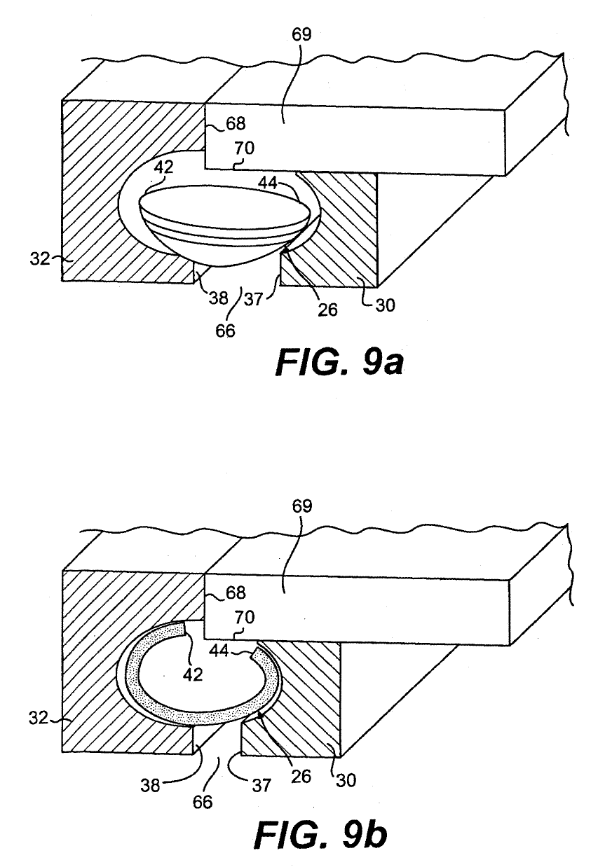

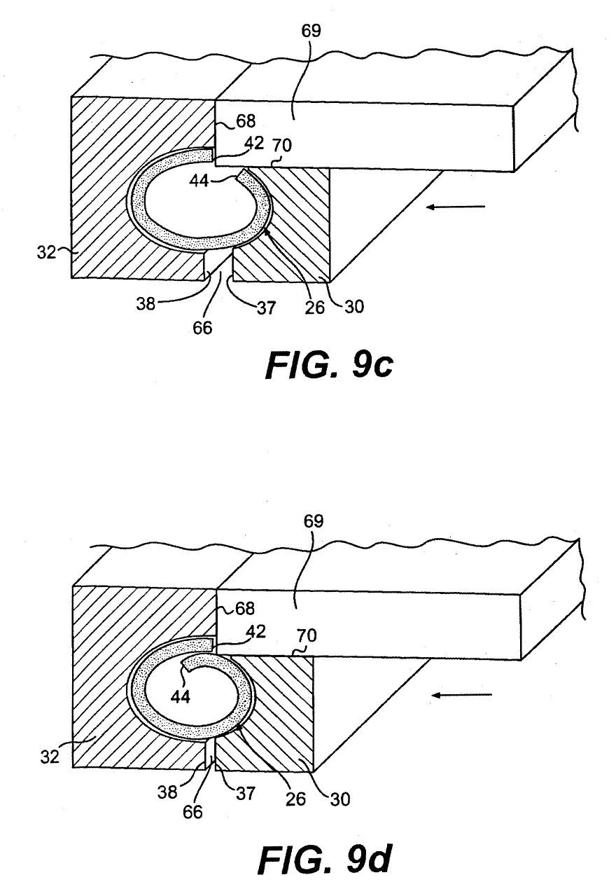

Referring to FIG. 9(a), another embodiment of the seal contact surfaces is shown. Here, the seal folding operation is again, performed by seal contact surfaces 37 and 38 of folding members 30 and 32, respectively, forming a dynamic channel 66 therebetween. In one aspect, channel 66 further comprises a shoulder member 69 that can facilitate folding of sealing element 26.

Shoulder member can, in one aspect, act as a seal stop to guide one edge of sealing element 26 under an opposed edge of the sealing element. For example, shoulder member 69 can be fixed in relation to folding member 32. In addition, folding member 30 can move relative to shoulder member 69. It should be appreciated, however, in other embodiments, shoulder surface 68 need not be fixed to contact surface 38, nor must shoulder member 69 be in contact with folding member 30.

In one aspect, shown in FIG. 9(a), shoulder member 69 has a shoulder or seal stop surface 68 and a seal guiding surface 70. Referring now to FIGS. 9(b)-(d), as the seal contact surfaces converge in relation to one another, seal contact surfaces 37 and 38 come in contact with seal edges 42 and 44 of sealing element 26. Upon further convergence, seal edges 42 and 44 slide up the respective seal contact surfaces and begin to curl back toward the center of dynamic channel 66. Seal edge 44 slides along guiding surface 70 of shoulder member 69, while seal edge 42 abuts seal stop surface 68 of shoulder member 69, preventing further movement of edge 42 in relation to contact surface 38. As convergence continues, shoulder member 69, thus, serves to guide seal edge 44 under seal edge 42.

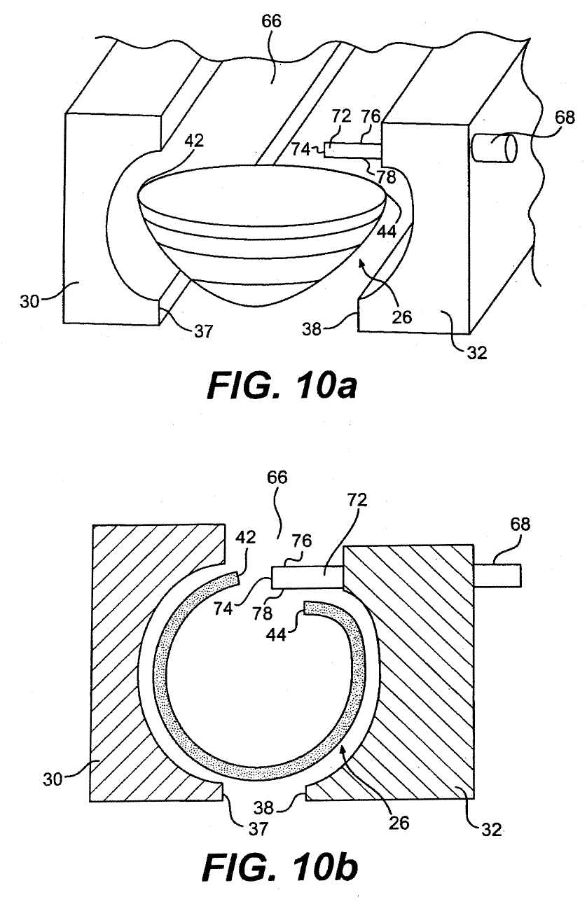

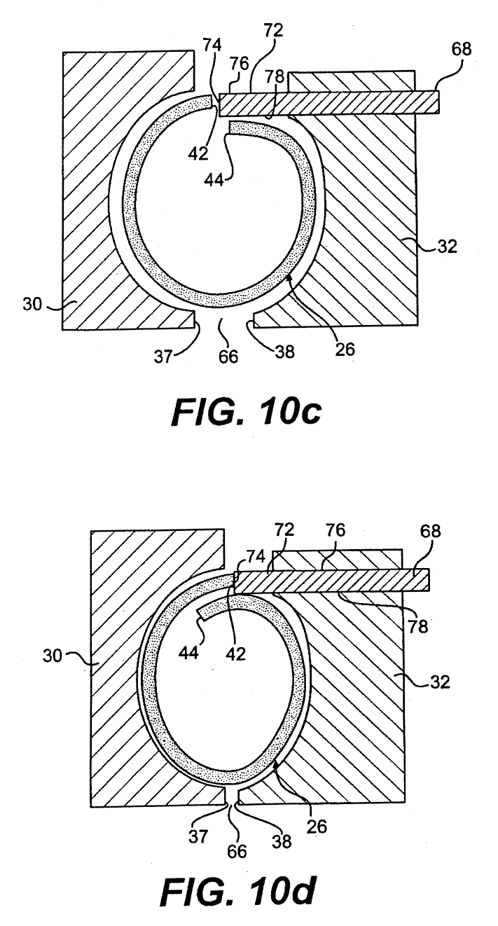

Referring now to FIG. 10(a), another embodiment of the seal loader apparatus is shown. In one aspect, there is a slidable pin 72 positioned in an aperture in folding member 32. The pin protrudes from seal contact surface 38 and extends into dynamic channel 66, defined by opposing seal contact surfaces 37 and 38. In use, pin 72 can contact sealing element 26 and direct one edge of the sealing element under or over another edge. As the seal contact surfaces converge, the pin can be withdrawn from channel 66 to permit further convergence and folding of sealing element 26.

In one aspect, pin 72 can extend through seal folding member 32 where a distal end 74 of pin 72 extends into channel 66 and a proximal end 68 of the pin extends out the opposite side of the seal folding member. In one aspect, the proximal end 68 of pin 72 allows user control of the pin. For example, as the folding members converge, the pin can be withdrawn by a user. Conversely, a user can partially fold the sealing element and then move the pin into channel 66 to direct one side of the sealing element under another side. In another aspect, the pin can be biased in the inserted configuration. As the folding members converge, folding member 32 can contact distal end 74 and move the pin out of the channel 66.

Referring now to FIGS. 10(b)-(d), as the seal contact surfaces converge in relation to one another, seal contact surfaces 37 and 38 come in contact with seal edges 42 and 44 of sealing element 26. Upon further convergence, seal edges 42 and 44 slide up the respective seal contact surfaces and begin to curl back toward the center of dynamic channel 66, seal edge 44 sliding along a downward facing surface of pin 72 while seal edge 42 abuts distal end 74 of pin 72, preventing further movement of edge 42 in relation to contact surface 37. As convergence continues, and as a result of the force seal edge 42 places on slidable pin 72, pin 72 is ejected from the channel while serving to guide seal edge 44 under seal edge 42.

In another embodiment, pin 72 can protrudes through folding member 30 at a lower height than described above (not illustrated), for example, pin 72 can extend through the curved surface of seal contact surface 38. Instead of seal edge 42 abutting distal end 74 of slidable pin 72 during seal contact surface convergence, edge 42 can curl over pin 72 and slide along upward facing surface 76. As convergence continues and slidable pin 72 is removed from dynamic channel 66.

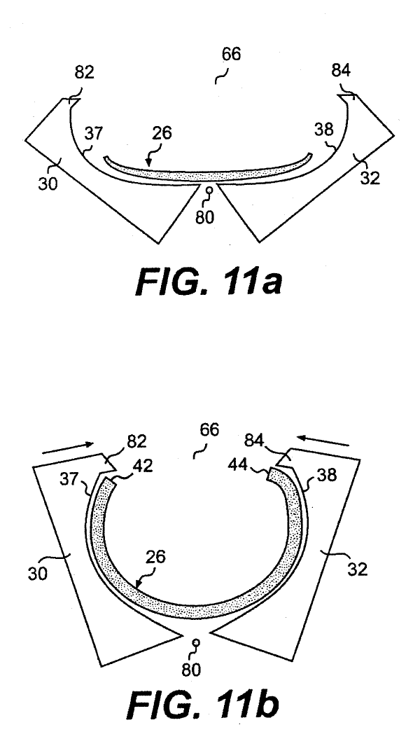

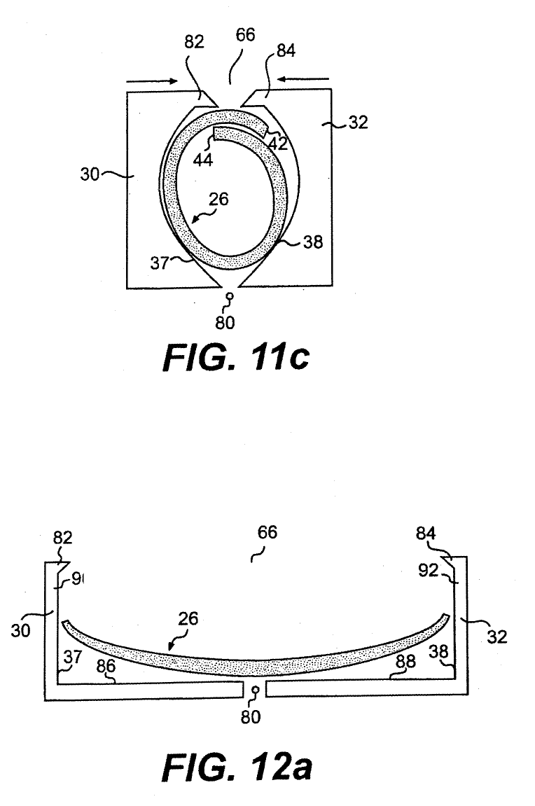

Referring now to FIG. 11(a), another embodiment of the seal folding apparatus is described. Here, folding members 30 and 32 can be maintained in hinged or pivotal relation, sharing a common pivotal axis 80 positioned generally parallel to a longitudinal axis of dynamic channel 66. Again, in one aspect, seal contact surfaces 37 and 38 are curved. In other embodiments, however, the contact surfaces can be flat or linear or exhibit some other contour. As shown in FIG. 11(a), seal contact surface 37, 38 are facing upwards and an unfolded, or substantially unfolded, sealing element can be placed therebetween, above the pivotal axis. The seal contact surfaces can converge through the rotation of one or both surfaces about pivotal axis 80.

Seal contact surfaces 37 and 38 can also incorporate projecting shoulders 82 and 84, respectively, at the edges of the contact surfaces away from pivotal axis 80. In one aspect, the projecting shoulders can be angled or curved so as to aid in guiding the sealing element edges back toward the center of dynamic channel 66 during seal contact surface convergence. In another embodiment, shoulders 82 and/or 84 can be configured to act as a seal stop to limit movement of the sealing element relative to one of the folding members.

Referring now to FIGS. 11(b) and (c), as the seal contact surfaces converge in relation to one another, seal edges 42 and 44 can slide along the respective seal contact surfaces and begin to curl back toward the center of dynamic channel 66. Upon further convergence, the seal edges can slide across shoulders 82 and 84, respectively, and overlap, leaving the sealing element in the prolate spheroid shape previously discussed.

It should also be appreciated, in other embodiments, features incorporated into the seal contact surface configurations discussed in relation to FIGS. 7(a)-10(d), intended to, among other things, ensure proper seal overlap and an adequate fold, can also be utilized in this configuration. For example, contact surface 37 can exhibit a different radius of curvature and/or length compared with contact surface 38.

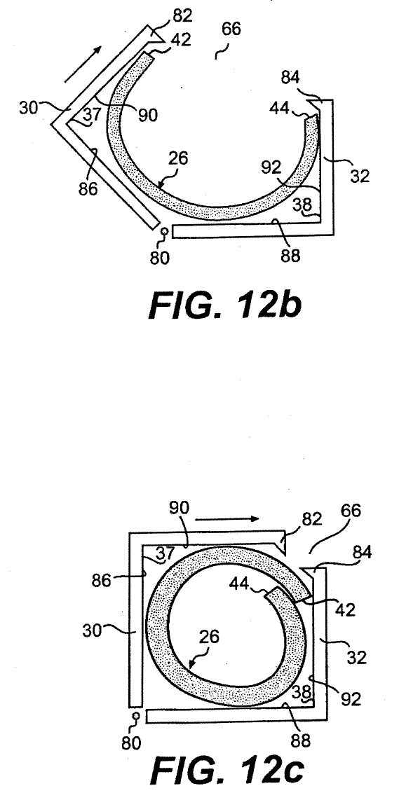

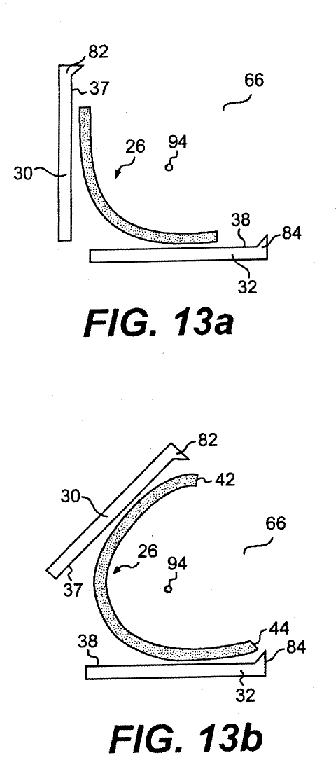

Referring now to FIG. 12(a), another seal contact surface configuration is depicted. As in FIGS. 11(a)-(c), folding members 30 and 32 can be maintained in hinged or pivotal relation, sharing a common pivotal axis 80 positioned between adjacent ends of the folding members 30, 32 and generally parallel to a longitudinal axis of dynamic channel 66. However, rather than the curved contact surfaces shown in FIGS. 11(a)-(c), contact surfaces 37 and 38 are “L-shaped,” each comprising two flat portions, a base portion and a wall portion. In one aspect, the seal contact surfaces can be positioned such that, when oriented as shown in FIG. 12(a), an unfolded, or substantially unfolded, sealing element can be placed atop the two base portions, 86 and 88, between the two wall portions, 90 and 92, and above pivotal axis 80. In another aspect, the seal contact surfaces converge through the rotation of folding arm 30 about pivotal axis 80.

Wall portions 90 and 92 can also incorporate shoulders 82 and 84, respectively, at the uppermost edge of the contact surfaces. In one aspect, the shoulders can be angled or curved so as to aid in guiding the sealing element edges back toward the center of dynamic channel 66 during seal contact surface convergence.

Referring now to FIGS. 12(b) and (c), as folding member 30 pivots about pivotal axis 80 and the seal contact surfaces converge in relation to one another, seal edges 42 and 44 slide along the respective seal contact surfaces and begin to curl back toward the center of dynamic channel 66. Upon further convergence, the seal edges slide across shoulders 82 and 84, respectively, and overlap, leaving the sealing element in the prolate spheroid shape previously discussed.

It should also be appreciated, in other embodiments, features incorporated into the seal contact surface configurations discussed in relation to FIGS. 7-11, intended to, among other things, ensure proper seal overlap and an adequate fold, can also be utilized in this configuration. For example, contact surface 37 can have a shoulder or lip formed therein such that, during contact surface convergence, seal edge 42 is prevented from movement in relation to seal contact surface 37 and seal edge 44 can be more easily guided under seal edge 42.

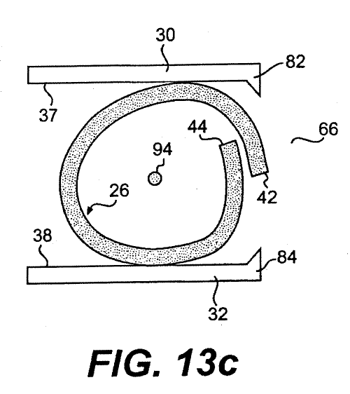

Referring now to FIG. 13(a), another seal contact surface configuration is depicted that provides rotation of at least one of the folding members around channel 66. Initially, in one aspect, seal contact surfaces, 37 and 38, of folding members 30 and 32, respectively, are oriented such that the contact surfaces are positioned at an angle with respect to one another (e.g., perpendicular) and an unfolded, or substantially unfolded, sealing element can be placed in dynamic channel 66, defined by the two contact surfaces. In this embodiment, seal contact surfaces 37 and 38 are flat or linear. The contact surfaces could, however, be curved or exhibit some other contour.

Folding member 30 can rotate about a rotational axis 94, such that the folding members move from a first position for receiving a substantially unfolded sealing element into a second position in which the folding members substantially oppose one another. Unlike the hinged contact surface pivoting described above, here, folding member 30 is rotated about a rotational axis, 94, that is parallel to the longitudinal axis of dynamic channel 66 and co-planar with one or neither of folding members 30 or 32. For example, in FIG. 13(a), rotational axis 94 is depicted as lying within dynamic channel 66, parallel to the longitudinal axis of the channel, and co-planar with neither folding member 30 or 32. In other embodiments, however, the rotational axis could be similarly parallel to the longitudinal axis of the channel but co-planar with folding member 32. For example, the axis could travel through the midpoint of folding member 32.

It should also be appreciated, in other embodiments, as opposed to only folding member 30 rotating about rotational axis 94, both folding members 30 and 32 can rotate about rotational axis 94. In another aspect, seal contact surfaces 37 and 38 can include a shoulder or lip formed therein such that, during contact surface convergence, sealing element 26 is prevented from movement in relation to at least one of contact surfaces 37, 38.

Additional features can also be incorporated into the seal loading apparatus to improve its functionality. For example, while movement of the folding members is generally described as directed by a user, in another embodiment the folding members can be spring loaded. Release or triggering of a spring can move one or more of the folding members and fold the sealing element. In such an embodiment, the user, prior to insertion of the sealing element, can position the folding members such that an unfolded, or substantially unfolded, sealing element can be placed therebetween. When the seal is to be folded, the user can manipulate an actuator, such as a switch or button, to release the spring and actuate the folding members. Using a mechanical force to actuate the folding members can provide a predetermined and repeatable amount of force, reducing the risk of the user exerting too little or too much force on the sealing element.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A seal loading apparatus for temporarily folding a surgical sealing element in a configuration for insertion into a lumen of a delivery device comprising:
a housing having at least one opening;
a first folding member having a first seal contact surface;
a second folding member having a second seal contact surface;
at least one of the first and second seal contact surfaces adapted to selectively move in relation to one another from a first position to a second position, defining a dynamic channel therebetween;
wherein the first and second seal contact surfaces are adapted to fold the hemostatic sealing element into a substantially prolate spheroidal shape when moved from a first position to a second position;
in the first position, the contact surfaces being spaced sufficiently for insertion of the sealing element in an unfolded, or substantially unfolded, configuration; and
in the second position; the contact surfaces being spaced such that at least a proximal or distal end of the seal has a cross sectional shape and size capable of at least partial insertion into the lumen of a delivery device.

2. An apparatus according to claim 1, wherein the first and second folding members are movably mated in relation to one another.

3. An apparatus according to claim 1, wherein the first and second folding members are independently controllable.

4. An apparatus according to claim 1, wherein the first and second folding members are configured as jaws.

5. An apparatus according to claim 1, wherein the first and second seal contact surfaces are maintained in substantially parallel relation and converge as they move from the first position to the second position.

6. An apparatus according to claim 1, wherein at least one of the first and second folding members rotates on an axis that is parallel to the longitudinal axis of the channel.

7. An apparatus according to claim 1, wherein at least one of the first and second folding members rotates on an axis that is not coplanar with at least one of the first or second folding members.

8. An apparatus according to claim 1, wherein one of the first and second seal contact surfaces has a higher coefficient of friction compared to the other of the first and second seal contact surfaces.

9. An apparatus according to claim 1, further comprising a seal stop surface to inhibit movement of the seal with respect to at least one of the first and second seal contact surfaces.

10. An apparatus according to claim 1, further comprising a window for viewing the channel.

11. An apparatus according to claim 1, wherein the apparatus is at least partially comprised of a transparent or translucent material.

12. An apparatus according to claim 1, further comprising a motion-limiting surface to prevent the seal contact surfaces from contacting one another.

13. An apparatus according to claim 1, further comprising a motion-limiting surface to limit the channel width to a distance equal to or greater than the delivery lumen.

14. A seal loading apparatus for temporarily folding a surgical sealing element in a configuration for insertion into a lumen of a delivery lumen comprising:
a flexible hemostatic sealing element; and
a loading device comprising:
a first folding member having a first seal contact surface;
a second folding member having a second seal contact surface;
at least one of the first and second seal contact surfaces adapted to selectively move in relation to one another from a first position to a second position, defining a dynamic channel therebetween;
in the first position, the contact surfaces being spaced sufficiently for insertion of the sealing element in an unfolded, or substantially unfolded, configuration; and
in the second position, the contact surfaces having a distance corresponding to the sealing element in a folded configuration,
wherein the first and second seal contact surfaces are adapted to fold the sealing element when moved from the first to the second position.

15. An apparatus according to claim 14, wherein the first and second folding members are independently controllable.

16. An apparatus according to claim 14, further comprising an abutment surface positioned so as to prevent passage of the sealing element through the channel.

17. An apparatus according to claim 14, wherein at least one of the first and second seal contact surfaces is not planar.

18. An apparatus according to claim 17, wherein one of the seal contact surfaces further comprises a seal stop surface adjacent to a curved portion thereof.

19. An apparatus according to claim 14, wherein the first and second seal contact surfaces have first and second curved portions.

20. An apparatus according to claim 19, wherein the first and second curved portions have different lengths.

21. An apparatus according to claim 19, wherein the first and second curved portions have different radii of curvature.

22. An apparatus according to claim 14, wherein one of the first and second seal contact surfaces has a shoulder formed thereon.

23. An apparatus according to claim 22, wherein the shoulder includes a surface generally transverse to at least one of the first and second seal contact surfaces.

24. An apparatus according to claim 14, further comprising a shoulder member fixed relative to the first contact surface and maintained in movable relation with respect to the second contact surface.

25. An apparatus according to claim 14, further comprising a pin extending through an aperture in the first folding member.

26. An apparatus according to claim 25, wherein the pin is slidably maintained in the aperture and extends into the channel in the first position.

27. A method for temporarily folding a surgical sealing element for insertion into a delivery lumen, comprising:
providing a loading apparatus comprising a first and second seal contact surface in a first position relative to one another, the first and second seal contact surfaces defining at least a portion of a dynamic channel;
inserting an unfolded, or substantially unfolded, flexible sealing element at least partially into the loading apparatus; and
folding the sealing element through relative movement of the seal contact surfaces from the first position to a second position.

28. The method according to claim 27, further comprising the step of inserting the folded sealing element into a delivery lumen.

29. The method according to claim 28, wherein the step of inserting the sealing element into the delivery lumen includes:
inserting a distal end of the delivery lumen into the loading apparatus and at least partially encapsulating an end of the sealing element within the distal end of the delivery lumen.

30. The method according to claim 28, wherein the sealing element, upon folding, has at least a portion thereof with an outer diameter equal to or greater than the inner diameter of the delivery lumen.

31. The method according to claim 27, wherein the step of folding includes folding the sealing element into a substantially prolate spheroidal shape.

32. A system for temporarily folding a surgical sealing element in a configuration for insertion into a lumen of a delivery lumen having a proximal and distal end and an opening shaped and sized for receiving a folded hemostatic sealing element, the system comprising:
a seal loading apparatus comprising a first seal contact surface and a second seal contact surface, wherein the first and second contact surfaces are configured to move between a first position and a second position relative to one another and defining a dynamic channel therebetween, in the first position, the contact surfaces being spaced sufficiently for insertion of the sealing element in an unfolded, or substantially unfolded, configuration, in the second position, the contact surfaces being spaced such that the dynamic channel has a cross-sectional width approximately equal to or greater than the outer diameter of the delivery lumen, the seal loading apparatus further comprising an abutment surface positioned to prevent passage of a hemostatic seal though the dynamic channel.

33. A system according to claim 32, wherein the first and second seal contact surfaces of the loading apparatus are movably mated in relation to one another.

34. A system according to claim 32, wherein the loading apparatus further comprises a window for viewing the channel.

35. A system according to claim 32, wherein the loading apparatus is at least partially comprised of a transparent material.

36. A system according to claim 32, wherein the channel, when the contact surfaces are in the second position, is sized such that the delivery lumen may be inserted therein.
